(19) United States
(12) Patent Application Publication
(10) Pub. No.: US 2024/0415404 A1
(43) Pub. Date: Dec. 19, 2024

(54) MULTI-ATTENUATOR NEUROLOGICAL ELECTRODE SYSTEM FOR MAGNETIC RESONANCE ENVIRONMENTS

(71) Applicant: RHYTHMLINK INTERNATIONAL, LLC, Columbia, SC (US)

(72) Inventors: James W. Kronberg, Columbia, SC (US); Harrison M. Floyd, Columbia, SC (US); Daniel E McCoy, Columbia, SC (US); Gabriel Orsinger, Columbia, SC (US); Brian Petree, West Columbia, SC (US)

(21) Appl. No.: 18/211,454

(22) Filed: Jun. 19, 2023

(51) Int. Cl. A61B 5/055; A61N 1/08

(52) U.S. Cl. CPC A61B 5/055; A61N 1/08; A61B 2562/222

(57) ABSTRACT

Disclosed are aspects of an electrode system and method that include an electrode, a connector, and a cable with one or more inline frequency filter modules comprising one or more inductors wired in series, and without any added capacitance. The one or more inline filter modules are placed along the cable and provide filtering of RF energy, thus minimizing the accumulation of heat at the electrode to patient connection. Further, in some aspects, the one or more inline filter modules are located at one or more specific locations along the cable, chosen through computer modeling and real-world testing, for minimum transfer of received RF energy to a patient's skin from the electrode.

### CROSS-REFERENCE TO RELATED APPLICATIONS

This application is a continuation-in-part of U.S. patent application Ser. No. 17/491,721, filed on Oct. 1, 2021, entitled “NEUROLOGICAL ELECTRODE SYSTEM FOR MAGNETIC RESONANCE ENVIRONMENTS”. Which is a continuation of and claims the benefit and priority of U.S. Pat. No. 11,160,483, filed on Sep. 15, 2020, entitled “NEUROLOGICAL MONITORING FOR CABLE MAGNETIC RESONANCE ENVIRONMENTS”, which further is a continuation of International Patent Application No. PCT/US2020/013514, filed on Jan. 14, 2020, entitled “NEUROLOGICAL MONITORING CABLE FOR MAGNETIC RESONANCE ENVIRONMENTS”, which further claims the priority and benefit to U.S. Provisional Patent Application No. 62/793,173, filed on Jan. 16, 2019, entitled “NEUROLOGICAL MONITORING CABLE FOR MAGNETIC RESONANCE ENVIRONMENTS” the contents of which are herein incorporated by reference.

### TECHNOLOGY FIELD

This disclosure relates to neurological monitoring and subsets such as intraoperative neurophysiological monitoring (IONM), occurring within magnetic resonance environments.

### BACKGROUND

EEG electrodes are small sensors used in neurological monitoring. These sensors are typically placed on the scalp to detect electrical signals generated by the firing of neurons in the brain. An EEG electrode is part of a system that includes the electrode, a cable, a connector, an amplifier and monitoring equipment. The electrode itself is often made of conductive materials, such as silver, gold, or tin, which allows for the efficient transfer of electrical signals. The electrodes are most often placed at locations on the scalp according to a standardized system, such as the International 10-20 system. This system divides the scalp into regions based on percentages of the total distance between anatomical landmarks. There are other systems available, that further set out placement locations based on statistical evidence. There may be anywhere from 19-25 electrodes, and in some instances, up to 256 electrodes or more applied to a patient.

To use the system, the electrode is attached to the patient and acquires electrical signals in the brain, or in certain applications may be adapted to stimulate nerves in the brain; the cable is attached to the electrode at one end and to an amplifier via the connector, and then to monitoring equipment.

If the cable is in the presence of a magnetic field oscillating at a radio frequency (RF), such as that generated by a Magnetic Resonance Imaging (MRI) machine, the cable tends to act as an antenna and conducts the radio frequency (RF) energy. The RF energy in the cable heats the cable and any electrically resistive material connected to it. If the cable is connected to an electrode attached to the skin of the patient, resistance heating at the skin-electrode interface may result in a burn injury.

MRI monitoring is a common hospital procedure utilized during IONM, thus procedures and precautions are taken around MRI machines to avoid such injuries. Ironically, the stronger the magnetic field and the higher the radio frequency, the better the image quality obtained from MR imagine but also the greater the resistance heating and the potential for burns.

Because of the danger of MRI burns to a patient who requires neurological monitoring and is to undergo MRI procedures, the electrodes are normally removed from the patient prior to the imaging procedure, and then re-attached afterwards. Attaching and re-attaching electrodes to patients is done by technicians, and the task is time-consuming and expensive. Moreover, the patient is not being monitored when undergoing the MRI procedure.

There are, however, electrode systems that may remain attached to the patient's head during MR imaging subject to conditions. These electrode systems are typically referred to as “MRI-conditional.” The conditions on use of these electrodes may include limits on the strength of the magnetic field of the MR imaging device and the time the patient may remain in the magnetic field attached these electrode systems. MRI conditional electrode systems may use different materials that respond less to magnetic fields, for example, tank filters (inductor-capacitor circuits) inserted into the electrode cables to block unwanted RF energy. Unfortunately, tank filters are frequency-specific, so they are not always effective in reducing heating when used in MRI machines. The need to tune these filters individually to the precise frequencies used in MRI also makes them relatively costly and labor-intensive to build.

As a result, there continues to be a need for better ways to avoid or minimize RF heating in electrode systems attached to the patient during IONM and magnetic resonance imaging.

### SUMMARY

According to its major aspects and briefly recited, it has been found that a combination of inductors and resistors inserted in-line at an optimal position in the cable of the electrode system forms a radio frequency filter that reduces heating and is less frequency-specific than a tank filter.

An aspect of the disclosure is that the components of the present in-line filter do not include tank filters with their need for precise tuning.

In some aspects, the techniques described herein relate to a neurological electrode system for use in magnetic resonance environments, including: an electrode included of conductive material for acquiring electrical impulses for neurological monitoring; a connector configured to connect to an amplifier; a cable, having a first end and a second end, the first end in electrical connection to the electrode, the second end in electrical connection to the connector; and one or more inline filter modules configured in series along the cable, the one or more inline filter modules including one or more inductors configured in a series, the one or more inline filter modules configured to limit heat generation observed at the electrode to skin interface from a radio frequency (RF) field of an MRI machine.

In some aspects, the techniques described herein relate to a method for using an electrode for neurological monitoring within magnetic resonance environments, including: providing an electrode included of conductive material for acquiring electrical impulses for neurological monitoring; providing a connector configured to connect to an amplifier; providing a cable, having a first end and a second end, the first end in electrical connection to the electrode, the second end in electrical connection to the connector; applying one or more inline filter modules configured in series along the cable, the one or more inline filter modules including one or more inductors configured in a series; and applying the electrode with the one or more inline filter modules to a radio frequency (RF) field generated by an MRI machine, the electrode configured to limit heat generation observed at the electrode to skin interface.

These and other aspects of the disclosure and their features and advantages will be apparent to those skilled in the art of neurological monitoring from a careful reading of the Detailed Description, accompanied by the following drawings.

### BRIEF DESCRIPTION OF THE DRAWINGS

FIG. 1 is a side view of an electrode system including an in-line RF filter module, according to an aspect of the disclosure;

FIG. 2 is an end view of a cross section of the in-line RF filter module of FIG. 1;

FIG. 3 is a side perspective view of a double-sided printed circuit board designed for enclosure by the in-line module of FIG. 1, showing an example of the components used therein, according to an aspect of the disclosure;

FIG. 4 is a plan view of an alternative, single-sided printed circuit board designed for enclosure by the in-line module of FIG. 1, showing an example of the components used therein, according to an aspect of the disclosure;

FIG. 5 is an electronic schematic diagram of the filter module according to an aspect of the disclosure;

FIG. 6 is a graph of RF power delivered to a patient's skin for three different magnetic resonance radio frequencies when using a simulated in-line filter module placed in one of various locations along a 240-millimeter cable of an electrode system, according to an aspect of the disclosure; and,

FIG. 7 is a graph of RF power delivered to a patient's skin at a magnetic resonance radio frequency of 128 MHz, such as used in 3-Tesla MRI machines, placed along cables measuring 240, 300, 500, 700 and 1000 millimeters in length, respectively.

FIG. 8 is an illustration of an electrode system including a plurality of in-line RF filter modules, according to an aspect of the disclosure;

FIG. 9 is an end view of a cross section of the in-line RF filter module of FIG. 8;

FIG. 10 is a side perspective view of a double-sided printed circuit board designed for enclosure by the in-line module of FIG. 1, showing an example of the components used therein, according to an aspect of the disclosure;

FIG. 11 is a plan view of an alternative, single-sided printed circuit board designed for enclosure by the in-line module of FIG. 1, showing an example of the components used therein, according to an aspect of the disclosure;

FIG. 12 is an electronic schematic diagram of the filter module according to an aspect of the disclosure;

FIG. 13 is an illustration of a first spacing option for the in-line RF filter modules, according to an aspect of the disclosure.

FIG. 14 is an illustration of a second spacing option for the in-line RF filter modules, according to an aspect of the disclosure.

FIG. 15 is an illustration of an electrode bundle including a plurality of individual electrode systems, according to an aspect of the disclosure;

FIG. 16 is an illustration of an electrode bundle in use on a patient;

FIG. 17 is an illustration of an electrode system including a plurality of in-line RF filter modules, according to an aspect of the disclosure; and

FIG. 18 is an illustration of an electrode system including a plurality of in-line RF filter modules, according to an aspect of the disclosure.

### DISCUSSION OF THE PRIOR ART

An examination of the prior art in this field showed many U.S. patents already exist, including U.S. Pat. Nos. 7,945,322; 8,116,862; 8,180,448; 8,200,328; 8,301,243; 8,311,628; 8,463,375, 8,649,857 and 9,061,139) all by the same inventors (Stevenson et al.) and having the same objective of creating implantable devices using tank circuits to block specific undesirable frequencies.

A tank circuit is the parallel combination of an inductor (unavoidably including some resistance) with a capacitor, which may be discrete such as a manufactured chip or film capacitor or may include other capacitance contributed by nearby objects such as traces or copper areas left on a printed-circuit board. It blocks a typically narrow frequency range centered on fc=1 I 2n (LC), its resonant center frequency, where fc is the frequency in hertz, L the inductance in henries, and C the capacitance in farads.

For example, an inductor with a value of 390 nanohenries (“L”) and a ten-picofarad capacitor (“C”) yield fc=80.6 megahertz, close to the FM broadcast band.

The effect of resistance in the tank is to change a parameter “Q,” which becomes lower as the resistance increases. High “Q” makes the tank a very effective barrier at fc, with performance falling off sharply as the frequency deviates from it. Low “Q” broadens the frequency response, but at the expense of performance close to fc.

Because it is difficult to control the values of inductors and capacitors precisely, and account for stray capacitance and the effect of magnetic materials in the environment around a tank circuit, some degree of individual adjustment is usually needed to make each tank resonate at the desired fc. This requires the use of tunable components, such as adjustable capacitors, which are far costlier than stock fixed-value ones. The tank must then be isolated from outside effects, which could affect its tuning. In production, this typically adds significant cost. As a further disadvantage, the tank will then block only that one frequency (and a narrow band of others near it) while having little or no effect at others.

The prime object of the invention, therefore, is to provide a barrier against RF energy in EEG electrode leads which avoids the disadvantages of tank circuits by taking a wholly different approach: eliminating the use of parallel capacitance; treating the full electrode, cable and connector together as an antenna-like system at all typically-used MRI radio frequencies; and in that system placing in an optimal position along each cable a plurality of filter modules comprising a non-resonant filter effective at more than one such frequency, the optimal position being that which causes a minimum amount of RF energy to be delivered to the skin of a patient in contact with the electrode thereby minimizing the danger of burns.

Another object is to provide this RF energy barrier using components able to be used safely in an MRI environment, in the sense of being “MRI conditional” with field strengths and other conditions specified as needed.

A third object is to provide the MRI-compatible barrier using only low-cost, widely-available, stock-valued components requiring no individual adjustment after assembly.

A fourth object is to provide the barrier in the form of a compact filter module which can be mounted in-line in the electrode cables and be safe for use in a medical environment.

A fifth object is to make such a module, and thereby the electrode system containing it, more tolerant of radio-frequency energy and robust against resistance heating than the prior-art electrode systems.

### DETAILED DESCRIPTION OF THE INVENTION

This disclosure is filed with the benefit of and priority to U.S. Pat. No. 11,160,483, the contents of which is incorporated herein in the entirety.

A computer model was developed for a set of neurological monitoring electrodes to evaluate RF pickup from an RF device. The model was developed using the commercially-available EZNEC+, Version 6.0 antenna modeling software-https://eznec.com/.

An “inductor” as defined herein is a passive electronic component

The term “configured” as utilized herein is defined as attached/attaching or connected/connecting, and may include an intermediary component, such as a wire, or other component such as solder or conductive adhesive, including also an insulator or other component for electrically insulating the configured component.

In one aspect, the electrodes, wires, connectors, and the patient's head are represented as parts of a radio-frequency receiving antenna. The patient's head is divided into nineteen conductive volumes, each with its own resistance and capacitance, to simulate the distribution of radio-frequency current through an extended, electrically resistive load via the skin effect. The external cables are represented by straight wires, dangling wires, or a loop that includes a capacitor representing a multi-electrode connector.

Loads, simulated by two 3000-ohm resistors, simulate the typical resistance between the skin and each electrode. Additional loads, each comprised of inductance and resistance, are modeled in a way permitting easy relocation along the wires to simulate filter modules placed in varying locations.

To simulate the rotating RF field around a patient undergoing MR Imaging, the “birdcage” coil used as an RF source in a typical MRI machine was modeled as a set of four interconnected source dipoles, each dipole being 90 degrees out of phase with the next.

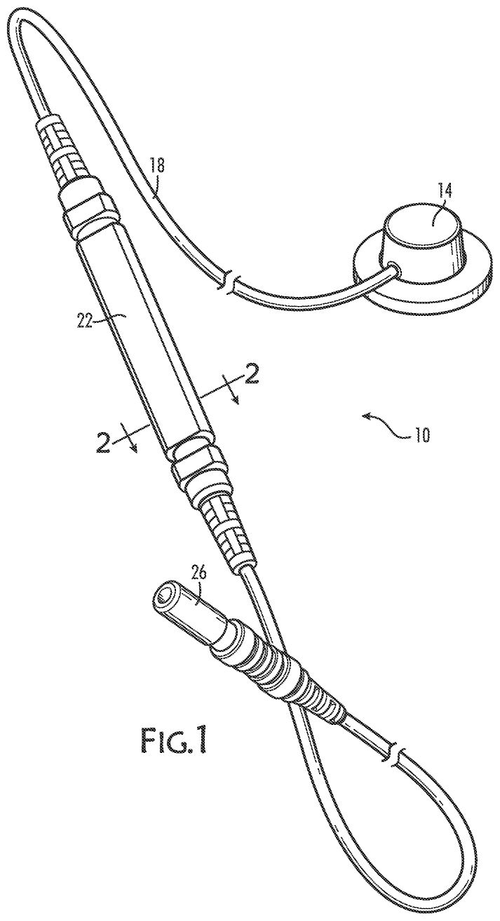

Referring now to FIGS. 1-5, FIG. 1 shows an electrode system 10 including an electrode 14, a cable 18 with an in-line filter module 22, and a connector 26. Cable 18 is in electrical connection with electrode 14 and with connector 26. Electrode 14 may be attached to the head of a patient along with other electrodes for neurological monitoring or other neurological procedure. Connector 26 along with other connectors of other cables are connected to an amplifier (not shown) to amplify the signals received from electrode 14 and which signals traveled through cable 18 and in-line filter module 22.

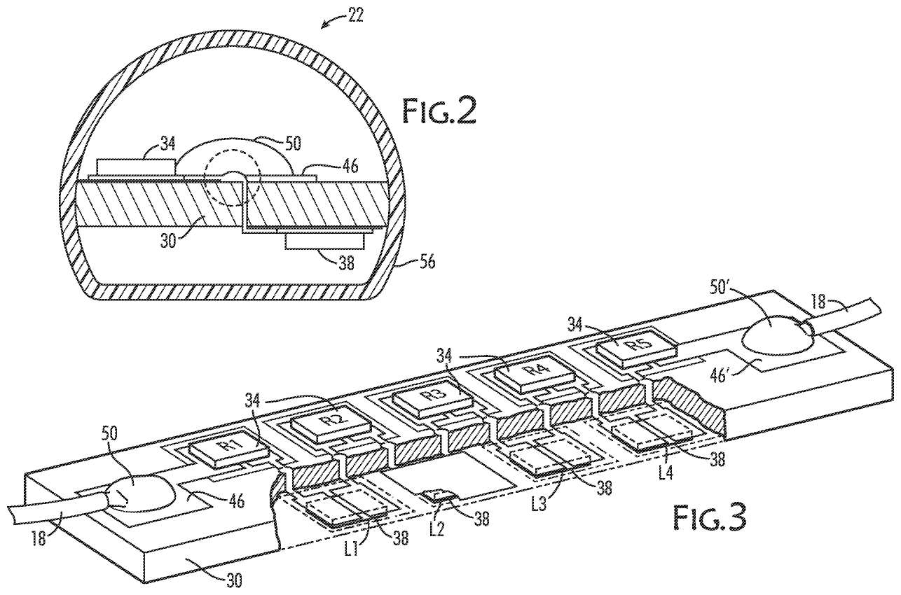

A cross-sectional view of in-line filter module 22, cut along line 2-2 in FIG. 1, is shown in FIG. 2. In-line module 22 includes a housing 56 made from a tough, electrically nonconductive and nontoxic polymer such as epoxy, silicone rubber, polyvinyl chloride, polyethylene or polypropylene. Housing 56 contains and protects a substrate 30, such as a small printed-circuit board, which is shown in perspective in FIG. 3, to which are attached plural resistors 34 alternatingly in series with plural inductors 38. Substrate 30 is inserted in-line in cable 18 so cable 18 is electrically connected to both ends of substrate 30 at contact pad 46, 46′, with solder, conductive epoxy, graphite-paste “wire glue” or other suitable connecting material 50, 50′. Substrate 30, and thereby in-line filter module 22 is thus in electrical connection with electrode 14 and connector 26.

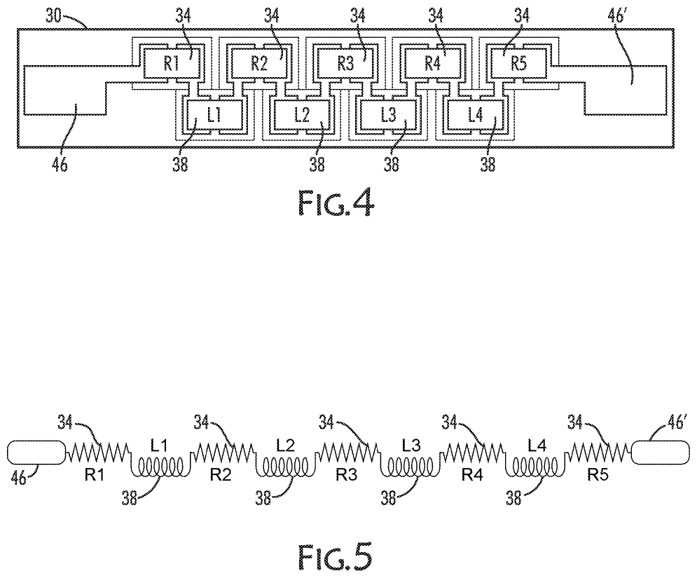

In FIGS. 3 and 4, resistors 34 and inductors 38 are shown as they might be mounted on two different types of printed circuit boards: double-sided in FIG. 3 and single-sided in FIG. 4. In each case the number of inductors “N” is four, so the number of resistors “N+1” in an alternating set: resistor 34, inductor 38, resistor 34, inductor 38, and so forth, with all resistors and inductors connected electrically in series. In FIG. 3, the inductors and resistors are placed on opposite sides and connected through vias, while in FIG. 4 all components are on the same side of the board. The latter approach simplifies construction, though at the cost of an increase in overall width.

In any manufacturer's series of standard miniature surface-mount inductors, those with higher inductance values have cores made of ferrite, a magnetic ceramic, while lower-valued ones use nonmagnetic ceramics such as porcelain or alumina. Typically, 390 nanohenries (0.39 microhenries) is the largest value currently made without a ferrite core.

Although comprised chiefly of iron oxide, ferrites come in many compositions optimized for different frequency ranges. They respond strongly to magnetic fields, both by experiencing physical force and by undergoing magnetic saturation which, if the ferrite is used in an inductor, will change the inductor's value. Accordingly, ferrite cores should be avoided in inductors meant for use in strong magnetic fields or near devices, such as MRI equipment, generating them.

In simulation, values of inductance found usable for the invention were in the range of one to two microhenries with an optimal value around 1.56 microhenries (1560 nanohenries). This value is easily achieved by connecting four ferrite-free, off-the-shelf 390-nanohenry miniature inductors electrically in series.

It is convenient for manufacturing, although otherwise not strictly necessary, to make inductors 38 all have the same nominal value and manufacturer's part number. For a total inductance of 1.56 microhenries, divided among four inductors as shown in FIGS. 3, 4 and 5, this nominal value, as just stated, is 390 nanohenries.

It should be stressed that nominal values include some error and are typically given with tolerances of ±1%, ±5% or the like, so an inductor sold as “390 nanohenries±5%” might have an actual value lying anywhere from 370.5 to 409.5 nanohenries. Differences of this order are often critical to the correct operation of tank circuits, but in the present design should make little difference.

The nominal values of each component type most often manufactured, usually standardized among manufacturers, are known as stock values. 390 nanohenries is an example of such a stock value. It is possible that inductors with different stock values than 390 nanohenries may in some cases be found more convenient to use. For example, advances in miniature inductor technology may yield higher inductance values without using ferrite, thus permitting a needed value to be achieved using a smaller number of physically discrete inductors.

Inductors 38 should be physically spaced a small distance apart so their magnetic fields do not overlap significantly. Such overlap, and the resulting interaction between their fields, could change their effective total inductance. Spacing is conveniently achieved by setting them physically apart in an alternating arrangement with the resistors, as shown in FIGS. 3 and 4. Conveniently, the physically adjacent devices are then connected electrically, again alternating between inductors and resistors, as shown schematically in FIG. 5.

Such an alternating arrangement has the additional advantage of distributing the heat from RF power dissipation in the resistors as widely as possible along the length of the filter module, minimizing potential hot spots. For the latter reason, and since chip resistors are much less costly than miniature inductors, it is desirable—although not strictly necessary—to have one more resistor (“N+1”) than inductor (“N”) as shown in FIGS. 3, 4 and 5, thus distributing any generated heat more widely.

Resistors may be selected to have a cumulative resistance of up to 1000 ohms, thus remaining within the input requirements for reliable operation of most EEG amplifiers. To allow for resistance in the cable, connections and the inductors themselves, however, it is desirable to make the actual total resistance within the filter module lower. Depending upon the values of those other resistances, a total resistance as low as 1 ohm within the filter module may be found usable.

Just as with the inductors it is convenient for manufacturing, although otherwise not strictly necessary, to make resistors 34 all have the same nominal or stock value and manufacturer's part number.

For example, in a preferred embodiment a filter containing four (“N”) 390-nanohenry inductors built according to this invention would include five (“N+1”) resistors. Dividing 1000 ohms by five yields 200 ohms. The next few±1% stock resistor values below 200 ohms are 196, 191, 187, 182 and 180 ohms. One of these values, or possibly one still lower if other resistances in the system are expected to be high, should be selected and may then be optimized by a modest amount of experiment.

A concrete example of in-line filter module 22 according to the preferred embodiment thus includes five resistors 34 each having a resistance of 180 ohms, and four ferrite-free inductors each having an inductance of 0.39 microhenries, arranged in alternation and connected in series beginning and ending with a resistor 34. The complete module thus has a resistance of 900 ohms in series with 1.56 microhenries.

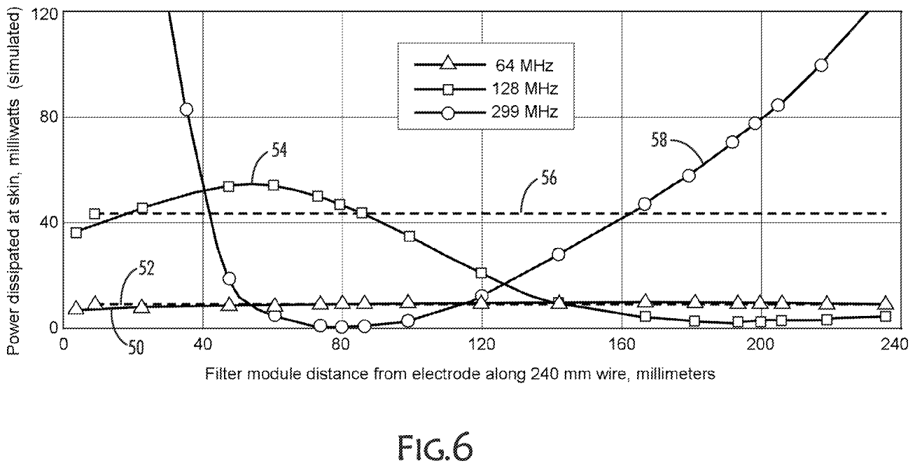

Simulation of the effectiveness of this in-line filter in a cable 18 between an electrode 14 and a connector 26, and exposed to three different commonly-used magnetic resonance frequencies, produced the response curves shown in FIG. 6 as a functions of the location of in-line module 22, embodying the concrete example given, along a cable 18 that is 240 mm long. The horizontal axis represents the distance along the wire from the electrode end, while the vertical axis shows the power delivered to a simulated skin resistance directly under the electrode. To avoid potential burns to the patient, as stated earlier, the prime object of the invention is to minimize this power.

Curve 50, with calculated data points indicated by triangles, shows the delivered power at 64 MHz while for comparison horizontal dashed line 52 shows a constant 8.8 milliwatts, the power with no filter module present. Given that the 240-millimeter wire occupies only 5% of the 4.65-meter wavelength of the 64-MHz RF energy, it functions very poorly as an antenna. Hence, the received and delivered power levels are low and adding the filter module makes little difference. RF burns have been of little concern with 1.5-Tesla MRI machines, which use 64 MHz as the RF frequency.

Increasing the magnetic field strength in an MRI machine improves the image quality and resolution, and to maintain resonance, the RF frequency is increased in proportion. Most new MRI machines operate at three Tesla, requiring a frequency of 128 MHz with a corresponding wavelength of 2.33 meters. Here the 240-millimeter wire occupies about 10% of the 2.33-meter wavelength, functioning much better as an antenna. This raises a definite concern of injury to a patient from RF energy.

Curve 54 shows the delivered power, while again for comparison dashed line 56 shows the power with no filter module present. As is readily seen, with no filtering the power at 128 MHz, 43.8 milliwatts, is nearly five times what was seen at 64 MHz.

At 128 MHz a filter module according to the preferred embodiment now has a strong effect on the delivered power, either raising or lowering it depending on the module's position. The region in which the module can be located to reduce the delivered power is surprisingly broad compared to the wire's length, and the amount of reduction at the minimum point is very substantial. For example, in a 240-millimeter wire, the minimum occurs with the module about 190 millimeters from the electrode, with delivered power of just 2.56 milliwatts: only 6% of the value without the module present.

Experimental MRI machines now in development use still stronger magnetic fields, typically of seven Tesla thus requiring an RF frequency of 299 MHz. Since at this frequency the 240-millimeter wire is nearly one-quarter wavelength, it picks up RF energy very efficiently.

Curve 58 shows a part of the resulting delivered power response, which extends far off the top of the chart at both ends. The power level without filtering, 3.18 watts, cannot be shown for comparison without expanding the graph and could easily be enough to cause serious injury to a patient. Installing the preferred embodiment of the filter module 80 millimeters from the electrode substantially reduces this power level to just 0.48 milliwatt: 0.015% of the unfiltered value.

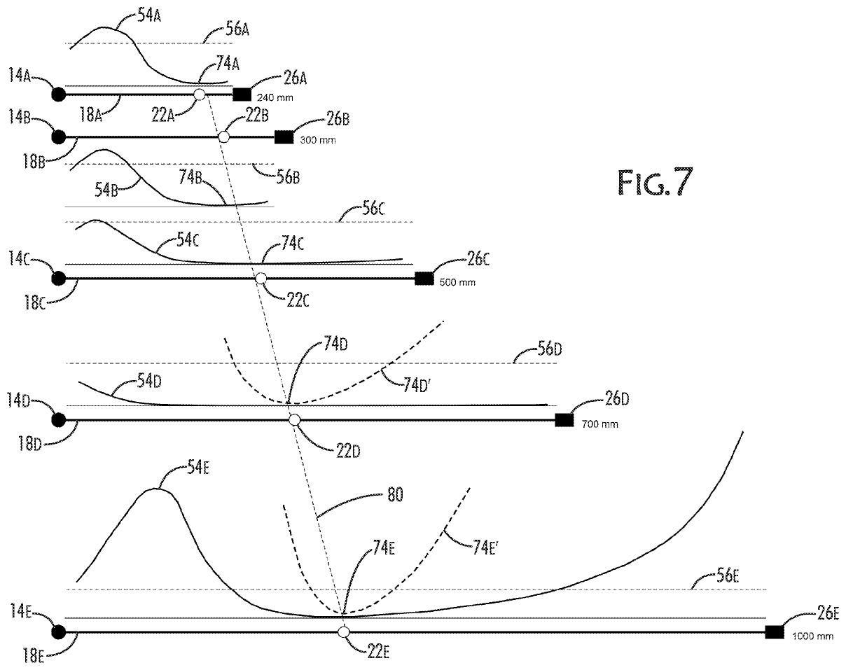

FIG. 7 shows the same curves for the three-Tesla frequency of 128 MHz only, for varying lengths of wire measuring 240, 300, 500, 700 and 1000 millimeters in length, respectively. Wires 18a-18e are depicted to scale, with electrodes 14a-14e at left and connectors 26a-26e at right.

Curve 54a reproduces curve 54 in FIG. 6 for the 240-millimeter wire showing power dissipated at the patient's skin as a function of the location of filter module 22, while dashed curve 56a reproduces line 56 showing the power with no filtering. Curves 54b, 54c, 54d and 54e, and dashed curves 56b, 56c, 56d and 56e, show the corresponding power curves and unfiltered power levels in the 300-, 500-, 700- and 1000-millimeter wires respectively.

As can be seen in FIG. 7, for each power curve a broad minimum appears, containing within it a point 74a, 74b, 74c, 74d or 74e at which the delivered power is minimized. For curves 74d and 74e, representing the delivered power for the 700- and 1000-millimeter wires, a dashed line 74d′ or 74e′ has been added magnifying a portion of each curve to show the minimum more clearly.

If filter modules 22a, 22b and so forth are drawn on each wire 18a, 18b and so forth in their correct positions for minimizing the delivered power, it can be seen from FIG. 7 that they fall very nearly on a straight line 80. The slight discrepancy may be due to the finite resolution (“segmentation”) of the EZNEC antenna modeling software.

For the concrete example described above, 900 ohms in series with 1.56 microhenries, used at a radio frequency of 128 megahertz, line 80 represents an optimum location for filter module 22 of LM=0.27 L+135, where L is the total length of the electrode system 10, LM is the distance from electrode 14 to the center of module 22, and all distances are expressed in millimeters. Similar formulas can probably be derived for filter modules containing other values of resistance and inductance.

It should be stressed, however, that since computer simulation required some simplifying assumptions the real-life measured curves will likely differ slightly from those shown. Optimization may then be obtained by modest experimentation that is well within the capability of those of ordinary skill in the art.

A series combination of inductors and resistors, without capacitors, when inserted into the cable of the electrode system in the form, for example, of an in-line filter module, forms an effective RF filter that reduces resistance heating at the patient's skin surface under and near the electrodes while being less frequency-specific than a tank filter, able to be made with stock off-the-shelf components, and requiring no individual tuning after assembly.

Optimizing in-line filter module 22 through experimentation on the number and value of the components, which are resistors 34 and inductors 38, and no capacitors; through favoring stock values for resistors 34 and inductors 38; and through favoring positions for in-line module 22 between the ends of cable 18, and generally toward the middle of a 250 centimeter cable; may provide an MRI cable 18 for electrode system 10 that has far fewer restrictions and is more tolerant of radio-frequency energy and robust against resistance heating than prior art electrode systems.

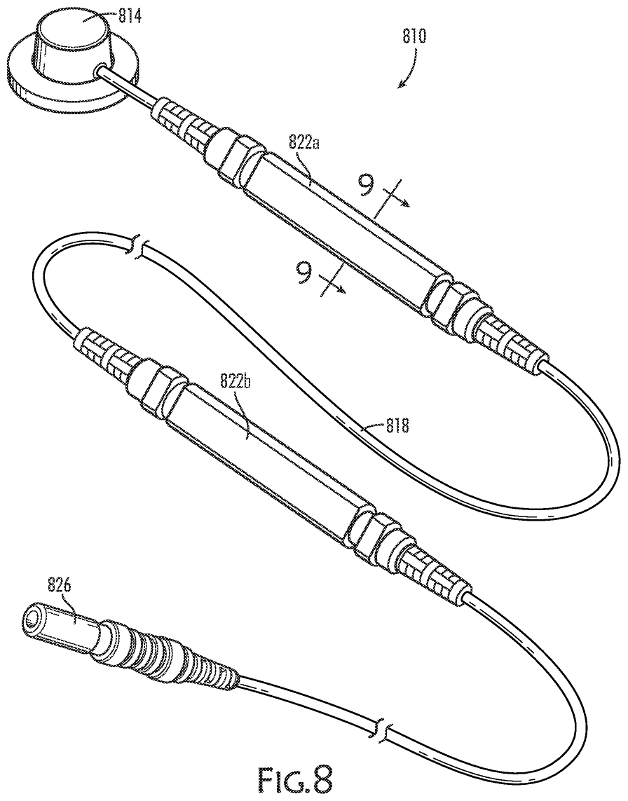

Continuing, in the example of FIG. 8, an electrode system 810 including an electrode 814 having conductive material (silver, gold, tin, etc.) for acquiring neurological electrical impulses, a cable 818 with one or more in-line filter module(s) 822, and a connector 826 is shown. The cable 818 is in electrical connection with the electrode 814 and with the connector 826. The connector 826 along with other connectors of other cables may be connected to an amplifier (not shown) to amplify the signals received from the electrode 814 (which signals travels through the cable 818 and the in-line filter modules 822).

In particular, as illustrated in FIG. 8, the in-line filter modules 822a, 822b are in series along the cable 818. Moreover, each of the in-line filter modules 822 include circuitry having one or more inductors in series (in contrast to FIG. 3, for example, which has resistors and inductors). In this way, the electrode 814 may be located at an electrode to skin interface (e.g., attached to the head of a patient along with other electrodes for neurological monitoring or other neurological procedure, such as that shown and described for FIGS. 1-7 and generally herein) in a magnetic resonance environment, and yet the electrode system 810 limits heat generation observed at the electrode 814 from the RF field of the magnetic resonance environment. More specifically, after about 15 minutes in a magnetic resonance environment, the electrode system 810 limits heating at the electrode to skin interface to less than about 7.0 degrees) (° centigrade (C), in worst-case scenario testing. In another embodiment, under normal operating procedures, the electrode system 810 limits heating at the electrode to skin interface to less than about 5.0° C.

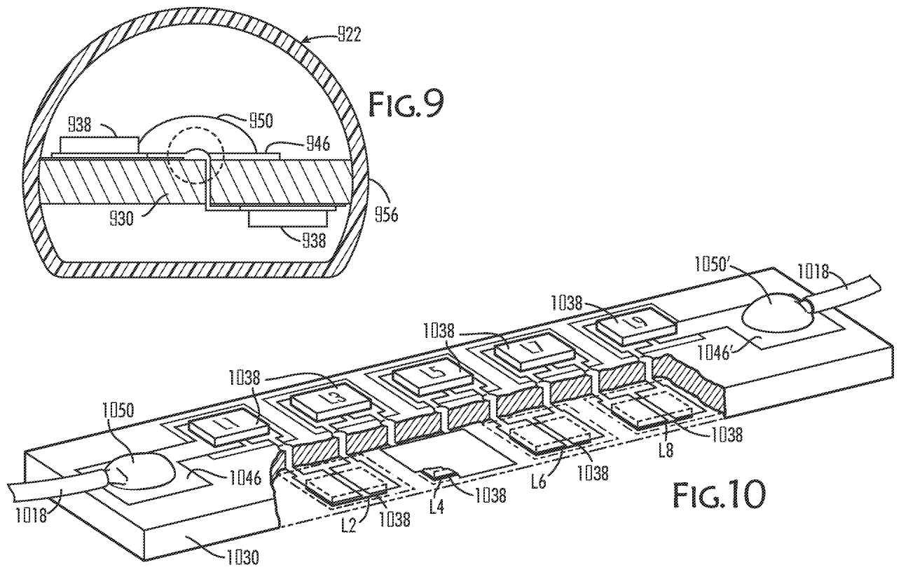

A cross-sectional view of an example in-line filter module, cut along line 9-9 shown in FIG. 8, is shown in FIG. 9. As illustrated in FIG. 9, an in-line module 922 includes a housing 956 made from a tough, electrically nonconductive and nontoxic polymer such as epoxy, silicone rubber, polyvinyl chloride, polyethylene or polypropylene. The housing 956 contains and protects a small printed-circuit board (PCB) 930, an example of which is shown in perspective in FIG. 10, to which are attached plural inductors 938 on a top, left side alternatingly in series with plural inductors 938 on a bottom, right side. The PCB 930 is inserted in-line in cable 918 (not shown) so cable 918 is electrically connected to both ends of the PCB 930 at the contact pads 946, 946′, with solder, conductive epoxy, graphite-paste “wire glue” or other suitable connecting material 950, 950′. The substrate 930, and thereby in-line filter module 922 is thus in electrical connection with electrode 914 and connector 926 (not shown).

In FIGS. 10 and 11, examples of inductors in series (in contrast to FIG. 3, for example, which has resistors and inductors) are shown as they might be mounted on two different types of a substrate or PCB: a double-sided example in FIG. 10 and single-sided example in FIG. 11. In each case all the inductors are connected electrically in series. In FIG. 10, the inductors 1038 can be placed on opposite sides and connected through vias (copper or other metal channels in the PCB), while in FIG. 11 the inductors 1138 are on the same (top) side of the PCB 1130. The latter approach simplifies construction, though at the cost of an increase in overall width. In either the example of FIG. 10 or FIG. 11, the inductors 1038, 1138 are physically spaced a small distance apart so their magnetic fields do not overlap significantly. The spacing is conveniently achieved by setting the inductors 1038, 1138 physically apart in an alternating arrangement, as shown in FIGS. 10 and 11. Conveniently, the physically adjacent sides of the inductors 1238 are then connected electrically, again alternating between inductors on one side (or on the top) and inductors on the other side (or on the bottom), as shown schematically in the third example of FIG. 12.

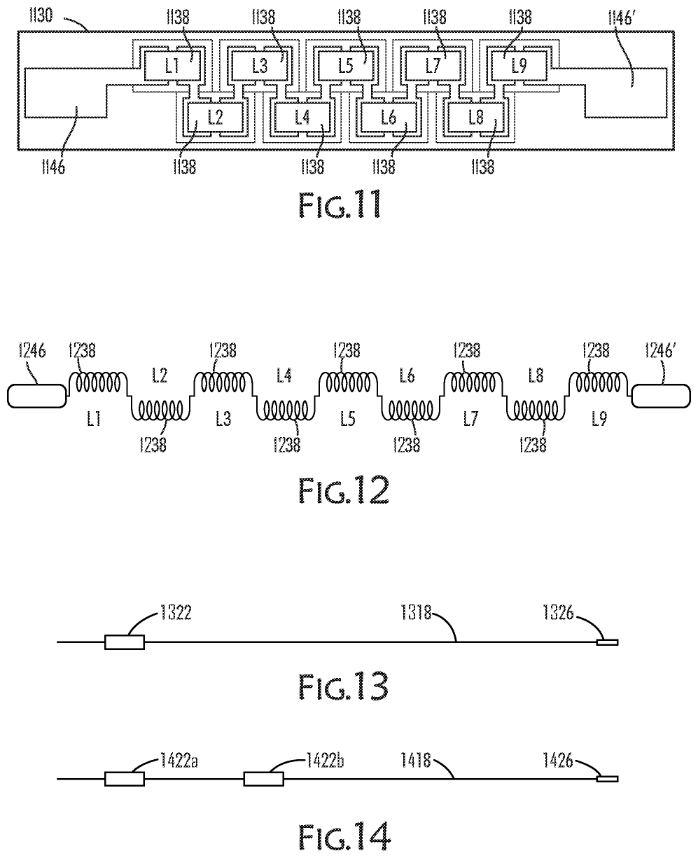

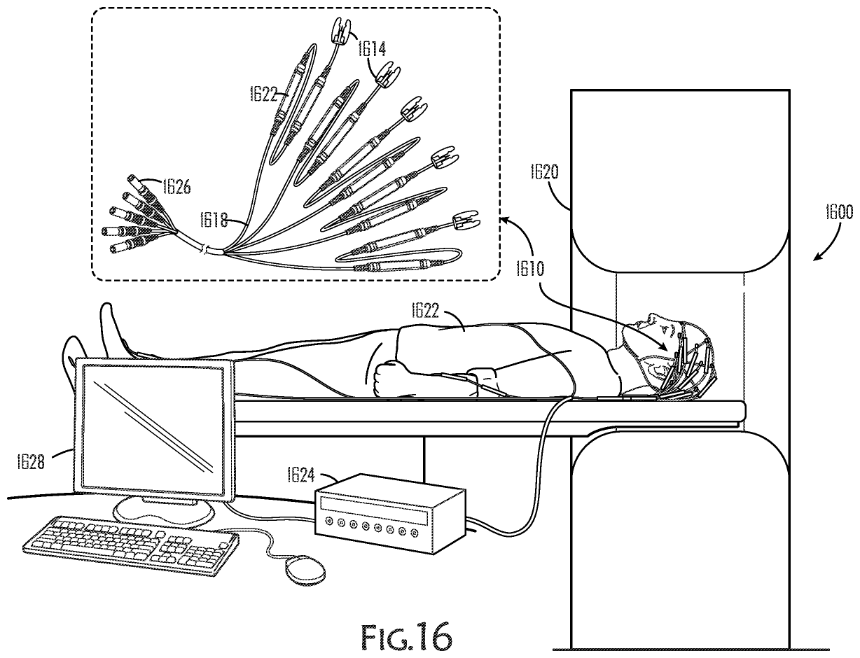

In FIGS. 13 and 14, examples of in-line filter modules having one or more inductors in series are shown as they might be spaced on a cable: a single in-line filter module example in FIG. 13 and a multiple in-line filter module example in FIG. 14. In each case the in-line filter module(s) are located in series along the cable. In FIG. 13, the single inline filter module 1322 is located between about 1.0 millimeters (mm) to about 75.0 mm from the electrode to skin interface, for example (not shown; best seen in FIG. 16) (any other in-line filter modules may follow similar spacing). In FIG. 14, the inline filter modules 1422a is located between about 25.0 millimeters (mm) to about 50.0 mm from the electrode to skin interface, and the inline filter module 1422b is located within about 500 mm of the electrode to skin interface (any other in-line filter modules may follow either/or spacing).

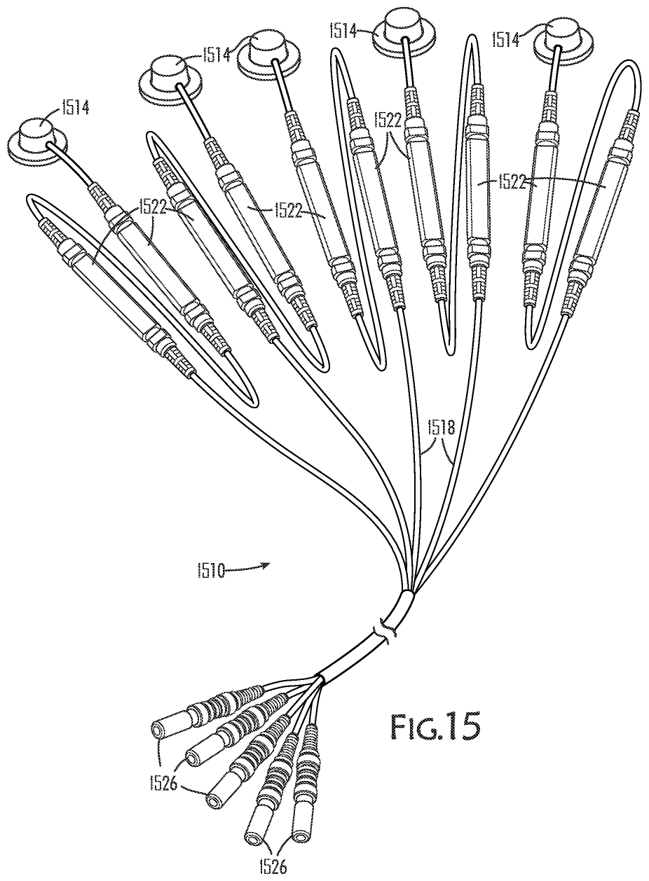

Continuing, in the example of FIG. 15, an electrode bundle 1510 including a plurality of individual electrode systems according to the present disclosure (for example, the electrode system 810 of FIG. 8) is shown. In particular, each individual electrode system of the electrode bundle 1510 includes its own electrode 1514, a cable 1518 with one or more in-line filter module(s) 1522, and a connector 1526. Each cable 1518 of each electrode system of the electrode bundle 1510 is in electrical connection with its electrode 1514 and with its connector 1526, and the in-line filter modules 1522 are in series along the cables 1518, and the cables 1518 are bundled together.

Continuing, in the example of FIG. 16, an electrode bundle 1610 (like the electrode bundle 1510 of FIG. 15, for example) is shown in use. The electrode bundle 1610 includes a plurality of individual electrode systems wherein each individual electrode system of the electrode bundle 1610 includes its own electrode 1614, a cable 1618 with one or more in-line filter module(s) 1622, and a connector 1626. The connectors 1626 are connected to an amplifier 1524 having a user interface device 1628 (e.g., monitor, tablet, computer, mobile device). The electrodes 1614 are attached to the head of a patient 1622 (e.g., located at an electrode to skin interface) (along with other electrodes for neurological monitoring or other neurological procedure, for example) and the patient 1622 is in a magnetic resonance environment 1600 produced by an MRI machine 1610. Despite the RF field of the magnetic resonance environment 1600, the electrode system 1610 limits heat generation observed at the electrodes 1614 according to the present disclosure.

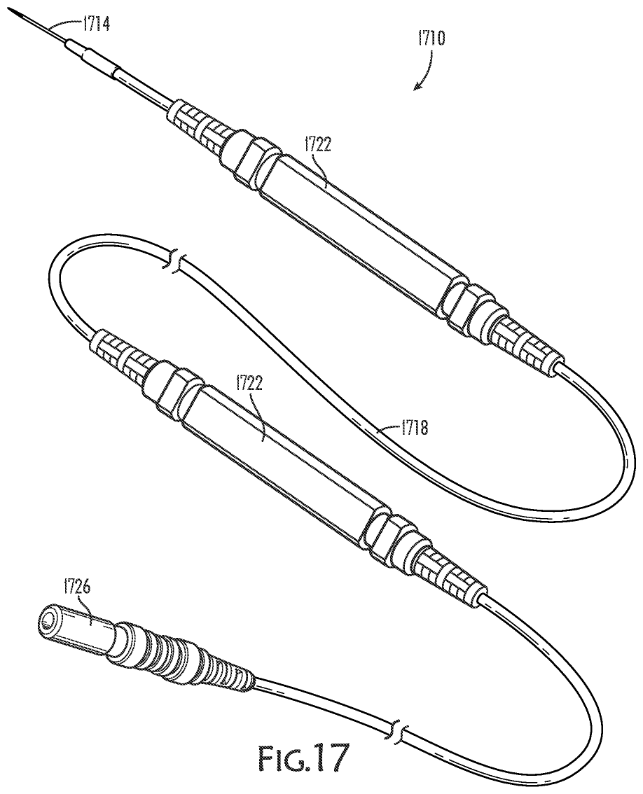

Continuing, in the example of FIG. 17, an electrode system 1710 including a subdermal needle 1714 for intraoperative neurological monitoring, for example, a cable 1718 with one or more in-line filter module(s) 1722, and a connector 1726 is shown. The cable 1718 is in electrical connection with the subdermal needle 1714 and with the connector 1726. The connector 1726 along with other connectors of other cables may be connected to an amplifier (not shown) to amplify the signals received from the subdermal needle 1714 (which signals travels through the cable 1718 and the in-line filter modules 1722). In particular, as illustrated in FIG. 17, the in-line filter modules 1722 are in series along the cable 1718.

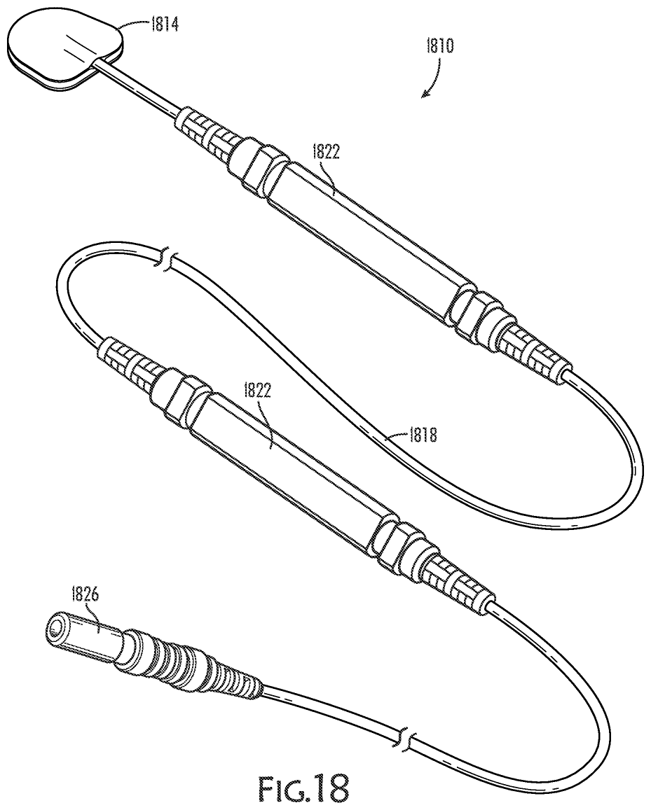

Continuing, in the example of FIG. 18, an electrode system 1810 including a sticky pad 1814 for use with a hydrogel, for example, a cable 1818 with one or more in-line filter module(s) 1822, and a connector 1826 is shown. The cable 1818 is in electrical connection with the sticky pad 1814 and with the connector 1826. The connector 1826 along with other connectors of other cables may be connected to an amplifier (not shown) to amplify the signals received from the sticky pad 1814 (which signals travels through the cable 1818 and the in-line filter modules 1822). In particular, as illustrated in FIG. 18, the in-line filter modules 1822 are in series along the cable 1818.

Aspects of the present disclosure may be further read in light of the following implementation clauses:

Clause 1. A neurological electrode system for use in magnetic resonance environments, comprising: an electrode comprised of conductive material for acquiring electrical impulses for neurological monitoring; a connector configured to connect to an amplifier; a cable, having a first end and a second end, the first end in electrical connection to the electrode, the second end in electrical connection to the connector; and one or more inline filter modules configured in series along the cable, the one or more inline filter modules comprising one or more inductors configured in a series, the one or more inline filter modules configured to limit heat generation observed at the electrode to skin interface from a radio frequency (RF) field of an MRI machine.

Clause 2. The system of clause 1, further comprising the cable having a length of up to 3,000 millimeters.

Clause 3. The system of clause 1, further comprising the one or more inline filter modules configured to limit the heat generation from the RF field at the electrode to skin interface to a maximum of 7° centigrade over ambient temperature during a 15-minute application of the RF field.

Clause 4. The system of clause 1, further comprising the RF field having either a 1.5 Tesla magnetic flux density or a 3.0 Tesla magnetic flux density.

Clause 5. The system of clause 1, wherein the neurological monitoring comprises intraoperative neurological monitoring (IONM).

Clause 6. The system of clause 1, wherein the electrode comprises a subdermal needle electrode, or a hydrogel electrode, or a metal plate electrode, or a suction electrode, or a flexible electrode.

Clause 7. The system of clause 1, further comprising the one or more inductors each being a chip inductor, spaced a small distance apart from one another, and wired in series along a printed circuit board.

Clause 8. The system of clause 1, further comprising each of the one or more inline filter modules having a polymeric housing.

Clause 9. The system of clause 1, further comprising the one or more inductors in series, each of the one or more inductors possessing similar rated inductance.

Clause 10. The system of clause 1, further comprising the one or more inductors having a total inductance between 1 and 2 microhenries.

Clause 11. The system of clause 1, further comprising the one or more inductors having a total inductance between 1370 and 1800 nanohenries.

Clause 12. The system of clause 1, wherein at least one of the one or more inline filters modules is located between 1 mm to 75 mm from the electrode, and at least a second one of the one or more inline filters is located within 500 mm of the electrode.

Clause 13. A method for using an electrode for neurological monitoring within magnetic resonance environments, comprising: providing an electrode comprised of conductive material for acquiring electrical impulses for neurological monitoring; providing a connector configured to connect to an amplifier; providing a cable, having a first end and a second end, the first end in electrical connection to the electrode, the second end in electrical connection to the connector; applying one or more inline filter modules configured in series along the cable, the one or more inline filter modules comprising one or more inductors configured in a series; and applying the electrode with the one or more inline filter modules to a radio frequency (RF) field generated by an MRI machine, the electrode configured to limit heat generation observed at the electrode to skin interface.

Clause 14. The method of clause 13, further comprising the cable having a length of up to 3,000 millimeters.

Clause 15. The method of clause 13, further comprising the one or more inline filter modules configured to limit the heat generation from the RF field at the electrode to skin interface to a maximum of 7° centigrade over ambient temperature during a 15-minute application of the RF field.

Clause 16. The method of clause 13, further comprising the RF field applied having either a 1.5 Tesla magnetic flux density or a 3.0 Tesla magnetic flux density.

Clause 17. The method of clause 13, wherein the neurological monitoring comprises intraoperative neurological monitoring (IONM).

Clause 18. The method of clause 13, wherein the electrode comprises a subdermal needle electrode, or a hydrogel electrode, or a metal plate electrode, or a suction electrode, or a flexible electrode.

Clause 19. The method of clause 13, further comprising the one or more inductors each being a chip inductor, spaced a small distance apart from one another, and wired in series along a printed circuit board.

Clause 20. The method of clause 13, further comprising each of the one or more inline filter modules having a polymeric housing.

Clause 21. The method of clause 13, further comprising the one or more inductors in series each of the one or more inductors possessing similar rated inductance.

What is claimed is:

1. A neurological electrode system for use in magnetic resonance environments, comprising:
an electrode comprised of conductive material for acquiring electrical impulses for neurological monitoring;
a connector configured to connect to an amplifier;
a cable, having a first end and a second end, the first end in electrical connection to the electrode, the second end in electrical connection to the connector; and
one or more inline filter modules configured in series along the cable, the one or more inline filter modules comprising one or more inductors configured in a series, the one or more inline filter modules configured to limit heat generation observed at the electrode to skin interface from a radio frequency (RF) field of an MRI machine.

2. The system of claim 1, further comprising the cable having a length of up to 3,000 millimeters.

3. The system of claim 1, further comprising the one or more inline filter modules configured to limit the heat generation from the RF field at the electrode to skin interface to a maximum of 7° centigrade over ambient temperature during a 15-minute application of the RF field.

4. The system of claim 1, further comprising the RF field having either a 1.5 Tesla magnetic flux density or a 3.0 Tesla magnetic flux density.

5. The system of claim 1, wherein the neurological monitoring comprises intraoperative neurological monitoring (IONM).

6. The system of claim 1, wherein the electrode comprises a subdermal needle electrode, or a hydrogel electrode, or a metal plate electrode, or a suction electrode, or a flexible electrode.

7. The system of claim 1, further comprising the one or more inductors each being a chip inductor, spaced a small distance apart from one another, and wired in series along a printed circuit board.

8. The system of claim 1, further comprising each of the one or more inline filter modules having a polymeric housing.

9. The system of claim 1, further comprising the one or more inductors in series, each of the one or more inductors possessing similar rated inductance.

10. The system of claim 1, further comprising the one or more inductors having a total inductance between 1 and 2 microhenries.

11. The system of claim 1, further comprising the one or more inductors having a total inductance between 1370 and 1800 nanohenries.

12. The system of claim 1, wherein at least one of the one or more inline filters modules is located between 1 mm to 75 mm from the electrode, and at least a second one of the one or more inline filters is located within 500 mm of the electrode.

13. A method for using an electrode for neurological monitoring within magnetic resonance environments, comprising:
providing an electrode comprised of conductive material for acquiring electrical impulses for neurological monitoring;
providing a connector configured to connect to an amplifier;
providing a cable, having a first end and a second end, the first end in electrical connection to the electrode, the second end in electrical connection to the connector;
applying one or more inline filter modules configured in series along the cable, the one or more inline filter modules comprising one or more inductors configured in a series; and
applying the electrode with the one or more inline filter modules to a radio frequency (RF) field generated by an MRI machine, the electrode configured to limit heat generation observed at the electrode to skin interface.

14. The method of claim 13, further comprising the cable having a length of up to 3,000 millimeters.

15. The method of claim 13, further comprising the one or more inline filter modules configured to limit the heat generation from the RF field at the electrode to skin interface to a maximum of 7° centigrade over ambient temperature during a 15-minute application of the RF field.

16. The method of claim 13, further comprising the RF field applied having either a 1.5 Tesla magnetic flux density or a 3.0 Tesla magnetic flux density.

17. The method of claim 13, wherein the neurological monitoring comprises intraoperative neurological monitoring (IONM).

18. The method of claim 13, wherein the electrode comprises a subdermal needle electrode, or a hydrogel electrode, or a metal plate electrode, or a suction electrode, or a flexible electrode.

19. The method of claim 13, further comprising the one or more inductors each being a chip inductor, spaced a small distance apart from one another, and wired in series along a printed circuit board.

20. The method of claim 13, further comprising each of the one or more inline filter modules having a polymeric housing.

21. The method of claim 13, further comprising the one or more inductors in series each of the one or more inductors possessing similar rated inductance.
